(19) United States
(12) Patent Application Publication
(10) Pub. No.: US 2002/0064777 A1
(43) Pub. Date: May 30, 2002

(54) Recombination repair gene, MIM, from arabidopsis thaliana

(76) Inventors: Tesfaye Mengiste, Durham, NC (US); Jerzy Paszkowski, Del Mar, CA (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/759,667

(22) Filed: Jan. 12, 2001

Related U.S. Application Data
(63) Continuation of application No. PCT/EP99/04984, filed on Jul. 14, 1999.

(30) Foreign Application Priority Data
Jul. 16, 1998 (GB) 9815485.9
Jan. 14, 1999 (GB) 9900760.1

(51) Int. Cl. C12Q 1/68; C07H 21/04; C12P 19/34; C12N 9/22

(52) U.S. Cl. 435/6; 435/91.2; 435/199; 536/23.2

(57) ABSTRACT

The present invention relates to DNA encoding proteins contributing to recombination repair of DNA damage in plant cells. The DNA sequence comprises an open reading frame encoding a protein characterized by an amino acid sequence having a 30% or more overall identity with SEQ ID NO: 3.

The present invention relates to DNA encoding proteins contributing to recombination repair of DNA damage in plant cells.

Cells of all organisms have evolved a series of DNA repair pathways which counteract the deleterious effects of DNA damage and are triggered by intricate signal cascades. Homologous recombination in plants stabilizes the genome by repairing damaged chromosomes simultaneously generating genetic variability through the creation of new genes and new genetic linkages. Repair of DNA damage by recombination is particularly significant for cells under exogenous and endogenous genotoxic stress because of its potential to remove a wide range of DNA lesions. The current understanding of genetic and molecular components underlying meiotic and somatic recombination and DNA repair in plants is limited. To be able to modify or improve DNA repair using gene technology it is necessary to identify key proteins involved in said pathways or cascades. Therefore it is the main object of the present invention to provide DNA comprising an open reading frame encoding such a key protein.

Within the context of the present invention reference to a gene is to be understood as reference to a DNA coding sequence associated with regulatory sequences, which allow transcription of the coding sequence into RNA such as mRNA, rRNA, tRNA, snRNA, sense RNA or antisense RNA. Examples of regulatory sequences are promoter sequences, 5′ and 3′ untranslated sequences, introns, and termination sequences.

A promoter is understood to be a DNA sequence initiating transcription of an associated DNA sequence, and may also include elements that act as regulators of gene expression such as activators, enhancers, or repressors.

Expression of a gene refers to its transcription into RNA or its transcription and subsequent translation into protein within a living cell.

The term transformation of cells designates the introduction of nucleic acid into a host cell, particularly the stable integration of a DNA molecule into the genome of said cell.

The present invention describes:

a DNA comprising an open reading frame encoding a protein characterized by an amino acid sequence having 30% or more identity with SEQ ID NO: 3,

the protein encoded by said open reading frame, and

a polymerase chain reaction, wherein at least one oligonucleotide used comprises a sequence of nucleotides which represents 15 or more basepairs of SEQ ID NO: 1

In particular the invention discloses:

DNA comprising an open reading frame encoding a protein comprising a stretch of 100 or more amino acids with 50% or more sequence identity to a stretch of aligned amino acids of a protein member of the SMC protein family;

DNA, wherein the open reading frame encodes a protein characterized by the amino acid sequence of SEQ ID NO: 3;

DNA characterized by the nucleotide sequence of SEQ ID NO: 1 or SEQ ID NO: 2;

DNA, wherein the open reading frame encodes a protein contributing to recombination repair of DNA damage in a plant cell;

DNA, wherein the open reading frame encodes a protein conferring hypersensitivity to treatment with methyl methanesulfonate (MMS);

DNA, wherein the open reading frame encodes a protein conferring hypersensitivity to treatment with X-rays, UV light or mitomycin C;

DNA, wherein the open reading frame encodes a protein with a NTP binding region followed by a first coiled coil region, a hinge or spacer, and a second coiled coil region followed by a C-terminal DA-box which harbours a Walker B type NTP binding domain; and

A method of producing said DNA, comprising

screening a DNA library for clones which are capable of hybridizing to a fragment of the DNA defined by SEQ ID NO: 1, wherein said fragment has a length of at least 15 nucleotides;

sequencing hybridizing clones;

purifying vector DNA of clones comprising an open reading frame encoding a protein with more than 40% sequence identity to SEQ ID NO: 3

optionally further processing the purified DNA.

DNA according to the present invention comprises an open reading frame encoding a protein characterized by an amino acid sequence having 30% or more overall identity with SEQ ID NO: 3. The protein characterized by SEQ ID NO: 3 is tracked down with the help of a T-DNA tagged Arabidopsis mutant showing hypersensitivity to methyl methanesulfonate (MMS). The mutant is also sensitive to X-rays, UV light and mitomycin C further supporting the notion that the corresponding wild type gene is involved in DNA damage repair. Finally, the mutant was found to be more sensitive to elevated temperatures than the wild type. Due to this multiply increased sensitivity, the mutant is called mim (sensitive to MMS Iradiation, Mitomicin C). The corresponding wild type gene is designated MIM. F1 hybrids between wild type plants and plants homozygous for the mutant mim gene do not show the mutant phenotype indicating a recessive mutation. Segregation of F2 seedling populations from a backcross to a wild-type indicate that the mutation is inherited as a recessive Mendelian trait.

Dynamic programming algorithms yield different kinds of alignments. In general there exist two approaches towards sequence alignment. Algorithms as proposed by Needleman and Wunsch and by Sellers align the entire length of two sequences providing a global alingment of the sequences resulting in percentage values of overall sequence identity. The Smith-Waterman algorithm on the other hand yields local alignments. A local alignment aligns the pair of regions within the sequences that are most similiar given the choice of scoring matrix and gap penalties. This allows a database search to focus on the most highly conserved regions of the sequences. It also allows similiar domains within sequences to be identified. To speed up alignments using the Smith-Waterman algorithm both BLAST (Basic Local Alignment Search Tool) and FASTA place additional restrictions on the alignments.

Within the context of the present invention alignments are conveniently performed using BLAST, a set of similarity search programs designed to explore all of the available sequence databases regardless of whether the query is protein or DNA. Version BLAST 2.0 (Gapped BLAST) of this search tool has been made publicly available on the internet (currently http://www.ncbi.nim.nih.gov/BLAST/). It uses a heuristic algorithm which seeks local as opposed to global alignments and is therefore able to detect relationships among sequences which share only isolated regions. The scores assigned in a BLAST search have a well-defined statistical interpretation. Particularly useful within the scope of the present invention are the blastp program allowing for the introduction of gaps in the local sequence alignments and the PSI-BLAST program, both programs comparing an amino acid query sequence against a protein sequence database, as well as a blastp variant program allowing local alignment of two sequences only. Said programs are preferably run with optional parameters set to the default values.

Sequence alignments of SEQ ID NO: 3 using commercially available computer programs based on well known algorithms for sequence identity or similarity searches reveal that a stretch of SEQ ID NO: 3 having 106 amino acids length shows up to 47% sequence identity to an aligned stretch of the S. pombe rad18 gene which is a member of the SMC (Structural Maintenance of Chromosomes) family of proteins. Though overall (global) identity or homology between SMC proteins is generally low, conserved motifs at the N- or C-terminal ends show significant identity or homology among SMC proteins and MIM, which has highest identity to a new subfamily of SMC proteins which includes RHC18 and rad18 also involved in DNA repair.

Overall (global) alignments of SEQ ID NO: 3 result in sequence identities lower than 30%O. Thus, the present invention defines a new protein family the members of which after overall alignment show 30% or higher amino acid sequence identity to SEQ ID NO: 3. Preferably overall amino acid sequence identity is higher than 55% or even higher than 70%. Most preferred are overall identities higher than 90%.

In a preferred embodiment of the present invention this new protein family comprises a stretch of 100 or more amino acids with 50% or more sequence identity to a stretch of aligned amino acids of a protein member of the SMC protein family such as the protein defined by SEQ ID NO: 3.

An example of DNA according to the present invention is described in SEQ ID NO: 1. The amino acid sequence of the protein encoded is identical to SEQ ID NO: 3. After alignment to the S. cerevisiae RHC18 amino acid sequence a stretch of 53 amino acids shows 54% sequence identity to the aligned RHC 18 sequence. Thus, according to the present invention, a protein family related to SMC proteins can be defined the members of which after alignment of a stretch of more than 50 amino acids length show 55% or higher amino acid sequence identity to SEQ ID NO: 3. Preferably the amino acid sequence identity is higher than 70% or even higher than 80%. When making multiple sequence alignments certain algorithms such as BLAST can take into account sequence similarities such as same net charge or comparable hydrophobicity/hydrophilicity of the individual amino acids in addition to sequence identities. Thus, they evaluate whether the substitution of one amino acid for another is likely to conserve the physical and chemical properties necessary to maintain the structure and function of the protein or is more likely to disrupt essential structural and functional features of a protein. Such sequence similarity is quantified in terms of of a percentage of positive amino acids, as compared to the percentage of identical amino acids. The resulting values of sequence similarities as compared to sequence identities can help to assign a protein to the correct protein family in border-line cases.

DNA encoding proteins belonging to the new protein family according to the present invention can be isolated from monocotyledonous and dicotyledonous plants. Preferred sources are corn, sugarbeet, sunflower, winter oilseed rape, soybean, cotton, wheat, rice, potato, broccoli, cauliflower, cabbage, cucumber, sweet corn, daikon, garden beans, lettuce, melon, pepper, squash, tomato, or watermelon. The following general method, can be used, which the person skilled in the art will normally adapt to his specific task. A single stranded fragment of SEQ ID NO: 1 or SEQ ID NO: 2 consisting of at least 15, preferably 20 to 30 or even more than 100 consecutive nucleotides is used as a probe to screen a DNA library for clones hybridizing to said fragment. The factors to be observed for hybridization are described in Sambrook et al, Molecular cloning: A laboratory manual, Cold Spring Harbor Laboratory Press, chapters 9.47-9.57 and 11.45-11.49, 1989. Hybridizing clones are sequenced and DNA of clones comprising a complete coding region encoding a protein with more than 30% overall sequence identity to SEQ ID NO: 3 is purified. Said DNA can then be further processed by a number of routine recombinant DNA techniques such as restriction enzyme digestion, ligation, or polymerase chain reaction analysis. Transformation of such genes into the mutant cell line mim leads to restoration of wild type levels of MMS, UV, and temperature resistance and wild type levels of root growth.

The disclosure of SEQ ID NO: 1 enables a person skilled in the art to design oligonucleotides for polymerase chain reactions which attempt to amplify DNA fragments from templates comprising a sequence of nucleotides characterized by any continuous sequence of 15 and preferably 20 to 30 or more base pairs in SEQ ID NO: 1. Said nucleotides comprise a sequence of nucleotides which represents 15 and preferably 20 to 30 or more base pairs of SEQ ID NO: 1. Polymerase chain reactions performed using at least one such oligonucleotide and their amplification products constitute another embodiment of the present invention.

### EXAMPLES

### Example 1

Cloning of the Gene Responsible for the mim Phenotype

The mim mutant phenotype is identified among a collection of Arabidopsis T-DNA insertion lines generated at the Institute National de la Recherche Agronomique (INRA), Versailles, France, as being sensitive to methyl methanesulfonate (MMS). Plants which die in the presence of 100 ppm MMS are found in a family designated CCK2. The test for MMS sensitivity is performed as described by Masson et al, Genetics 146: 401-407, 1997. Genomic DNA from the mutant is isolated according to the procedure described by Dellaporta et al, Plant Mol Biol Reporter 1: 19-21, 1983. Genomic DNA of the mutant Arabidopsis line is used to rescue DNA fragments flanking the right border of the inserted T-DNA using a modified protocol of the procedure described by Bouchez et al, Plant Mol Biol Reporter 14: 115-123, 1996. 2.5 μg of genomic DNA is digested with Pstl, ethanol precipitated and resuspended in H₂O. 2.5 μg of the vector pResc38 (Bouchez et al supra) is digested with Pstl and dephosphorylated with shrimp alkaline phosphatase. The phosphatase is heat inactivated and the vector DNA is ethanol precipitated and resuspended in H₂O. 2.5 μg of digested genomic DNA and 2.5 μg of digested and dephosphorylated vector DNA are mixed and ligated overnight at room temperature in a total volume of 100 μl with 10 units of T4 DNA ligase. The ligation mixture is precipitated with ethanol, rinsed 2 times with 70% ethanol, dried and dissolved in 5 μl of H₂0. 2 μl aliquots are used for electroporation of electrocompetent E.coli XL1-Blue cells (Stratagene) according to the manufacturer's instructions. Clones containing the T-DNA derived fragment and adjacent Arabidopsis genomic DNA are selected on plates with 50 mg/l kanamycin. Resulting single colonies are analyzed by isolation of plasmid DNA using QlAprep Spin Plasmid Kit (Qiagen) and digestion with Pstl. This procedure allows to isolate a fragment containing 3.7 kb of inserted T-DNA linked to 32 nt of adjacent Arabidopsis genomic DNA. Using a primer complementary to the T-DNA sequence 41 nucleotides from the right border and directed towards the plant flanking sequence (5′-GGTTTCTACAGGACGTAACAT-3′; SEQ ID NO: 4) the nucleotide sequence of the 32 nucleotides adjacent to the T-DNA derived fragment is determined and found to be 5′-CTG CAG ATC TGT TTA TGT TAA AGC TCT TTG TG-3′ (SEQ ID NO: 5).

### Example 2

Cloning of Wild-type MIM Gene Genomic and cDNA Sequences Wild-type MIM Gene

An oligonucleotide having the nucleotide sequence of the 32 bp Arabidopsis genomic DNA fragment mentioned in Example 1 is chemically synthesized. The oligonucleotide is end labelled with ³²P-γ-ATP using the forward reaction of T₄ polynucleotide kinase according to chapter 3 of Ausubel et al, 1994, “Current protocols in molecular biology”, John Wiley & Sons, Inc.) and used to probe a genomic DNA library (Stratagene) of wild type Arabidopsis thaliana ecotype Columbia in bacteriophage λ. Screening of the library is performed as described in chapter 6 of Ausubel et al, 1994, supra. Hybridization is performed as described by Church and Gilbert, Proc Natl Acad Sci USA 81: 1991-1995, 1984. Bacteriophage clones hybridizing to DNA probe are subjected to in vivo excision of plasmids according to Elledge et al, Proc Natl Acad Sci USA 88: 1731-1735, 1991, and Stratagene protocols. The 3 plasmid clones isolated are analyzed by sequencing which reveals that these overlapping clones lack the 5′end of the MIM locus. Therefore, the 5′ end of the longest genomic clone in pBluescript (pMIM3′8.1) contained on a 1.2 kb EcoRl-Sacl restriction fragment is labelled with ³²P by random oligonucleotide-primed synthesis (Feinberg et al, Anal Biochem 132: 6-13, 1983) and used as a probe to re-screen the genomic DNA library to identify clones containing the missing 5′ end of the MIM locus and overlapping with pMIM3′8.1. Sequencing and alignment of all overlapping clones reveals a continuous genomic DNA sequence for the MIM gene of 10156 bp comprising the wild-type MIM gene (SEQ ID NO: 1).

EcoRl Southern blot analysis of genomic DNA isolated from wild-type and mutant (mim) Arabidopsis using a 1.6 kb restriction fragment contained on pMIM3′8.1 and supposed to cover the T-DNA insertion site confirms that in the mutant (mim) genomic DNA the hybridizing restriction fragment in fact contains the T-DNA insertion.

In northern blot analysis using RNA extracted from callus, suspension culture cells, or flower buds of wild type plants, a transcript hybridizing to said fragment can be detected whereas no hybridizing fragment is detected using corresponding RNA samples extracted from mutant (mim) plant material.

MIM cDNA

A 4.2 kb EcoRl restriction fragment of genomic clone pMIM3′8.1 is subjected to ³²P random primed labeling (Feinberg et al, Anal Biochem 132: 6-13, 1983) and used to screen an Arabidopsis cDNA library as described by Elledge et al, Proc Natl Acad Sci USA 88: 1731-1735, 1991. 4 partial cDNA clones representing the same gene are identified; all lack the 5′ end of the predicted full-length cDNA (˜3.7 kb). Therefore, RT-PCR and 5′ RACE techniques are used to isolate the missing 5′ end of the MIM cDNA.

RT-PCR

Based on the known sequence of genomic DNA the following forward PCR primers (FP) are designed for RT-PCR:

| | |
|---|---|
| FP1: 5′-CTG GGT CGG GTT CGA TTC TGA G- 3′ | (SEQ ID |
| | NO:6) |
| FP2: 5′-GGT AAG AGT GCA ATA CTG ACT GC-3′ | (SEQ ID |
| | NO:7) |
| FP3: 5′-GCA GCT ATG CCG TTG TCC AAG TAG-3′ | (SEQ ID |
| | NO:8) |

Based on the sequence information available from the partial cDNA clones the following two specific reverse primers (SP) are designed:

SP1 (reverse): 5′-AAT GAC TCT GTC CCC TCC AAA TG-3′ (SEQ ID NO:9)
SP2 (reverse): 5′-ATG TTC GAG GTT ATG AAT CTT TG-3′ (SEQ ID NO:10)

Total RNA is extracted from actively dividing suspension culture cells using the Qiagen Plant RNeasy Kit. 5 μg of total RNA is reverse transcribed according to the manufacturer's instructions using AMV reverse transcriptase in the presence of deoxynucleotide mixtures (Boehringer Mannheim) using reverse primer SP1. The cDNA product is purified using High PCR Purification Kit (Boehringer Mannheim) followed by first round of PCR amplification using primers FP1 and SP2. The PCR product from the first round is diluted 1:20 and reamplified with FP2 and SP2. This PCR product is gel extracted and cloned into the pCR2.1 TA-cloning vector (Invitrogen). Sequencing and alignment with the genomic sequence reveal a 1.2 Kb cDNA towards the 5′ end still lacking the 5′ end.

PCR conditions include an initial denaturation step at 94° C. for 5 minutes followed by 25 cycles of denaturation at 94° C. for 30 seconds, annealing at 55° C. for 40 seconds, and extension at 72° C. for 1 minute, followed by a single final extension step of 7 minutes at 72° C.

5′ RACE

To identifiy the still missing 5′ portion of MIM cDNA the 5′ RACE (Rapid Amplification of cDNA Ends) technique is used. 2.5 μg of total RNA extracted from suspension culture cells of Arabidopsis is reverse transcribed using reverse primer RP1 (5′-GAC TCA GTT ATC CTG CGT TCG-3′; SEQ ID NO: 11). The resulting cDNA is 5′ end tailed with a homopolymeric A-tail using terminal transferase in the prescence of 2 mM dATP. The tailed cDNA is amplified using primers specific to the tailing oligonucleotide (Oligo dT-anchor primer 5′-GAC CAC GCG TAT CGA TGT CGA CTT TTT TTT TTT TTT TTV-3′; SEQ ID NO: 12; Boehringer Mannheim) and reverse primer RP2 (5′-GGA CAA CGG CAT AGC TGC ATC CAG-3′; SEQ ID NO: 13). The PCR product is diluted 1:20 and reamplified using PCR anchor primer (5′-GAC CAC GCG TAT CGA TGT CGA C-3′; SEQ ID NO: 14; Boehringer Mannheim) and reverse primer RP3 (5′-GGC AGC ACG CTG AGT CCC TCT CGC-3′; SEQ ID NO: 15). The specific PCR product is gel extracted and cloned into the pCR2.1 vector.

PCR conditions include a first round of PCR amplification of cDNA comprising a 5 minutes intial denaturation step followed by 25 cycles of denaturation at 94° C. for 30 seconds, annealing at 35° C. for 40 seconds, and extension at 72° C. for 40 seconds, followed by a final extension of 3 minutes at 72° C. The conditions of the second round of PCR are identical to the conditions used for RT-PCR. The amplification product is cloned into the pCR2.1 vector according to the manufacturer's instruction (Invitogen, TA-cloning kit).

### Example 3

Sequence Analysis and Alignments

The MIM cDNA (SEQ ID NO: 2) contains an ORF with the start codon spanning the nucleotide positions 73-75 and the stop codon spanning nucleotide positions 3238-3240. The ORF is capable of encoding a protein of 1055 amino acids with a predicted molecular mass of 121.3 kD and a theoretical pl of 8.3. Alignment with the genomic sequence shows 28 introns. The T-DNA in the mim mutant is inserted in the 22nd intron starting at nucleotide position 7835 of the wilde-type genomic sequence. The rescued sequence corresponds to the intronic sequence at positions 7804 to 7835 of the genomic sequence the beginning of which is marked by a Pstl restriction site (CTGCAG). The MIM ORF encodes a putative SMC-like protein (SEQ ID NO: 3) with an NTP binding domain at the amino terminus (amino acid positions 49 to 56), followed by the first coiled-coil region (amino acid positions 184 to 442), a hinge or spacer (amino acid positions 443 to 627), a second coiled-coil region (amino acid positions 628 to 909) followed by a conserved motif called the DA-box (amino acid positions 971 to 1007) which also harbours a Walker B type NTP binding domain. The structural organization of the MIM ORF is analysed for coiled-coil regions according to Lupas et al, Science 252: 1162-1164, 1991, and the coiled coil regions in the MIM ORF are delineated based on the probability of the encoded protein to form the coiled-coils.

Data base searching using the TFASTA program (Wisconsin Package Version 9.1, Genetics Computer Group (GCG), Madison, Wis.) reveals that the encoded protein has significant similarity to rad 18 of Schizosaccharomyces pombe and its homologue in Saccharomyces cerevisiae (RHC 18). The highest scoring homologues are S. pombe rad 18 and S. cerevisiae RHC18 genes (Lehmann et al, 1995) which show about 25% identity to overlapping stretches of more than 1000 amino acids length. The deduced MIM protein has also an overall identity of 20.6% to the RAD50 gene of yeast. Phylogenetic analysis (Wisconsin Package Version 9.1, Genetics Computer Group (GCG), Madison, Wis.) using the amino and carboxyl terminal sequences of the MIM ORF demonstrates that the encoded protein is distinct from other proteins belonging to the SMCs. The closest relatives in the database are S.pombe rad 18 and S.cerevisiae RHC18 genes (Lehmann et al, 1995).

A search in the SWISSPROT and NCBI databases using the BLAST program (Wisconsin Package version 9.1, Genetics Computer Group (GCG), Madison, Wis.) reveals that in a stretch of 121 aa surrounding the NTP binding site there is an identity of 42% when compared to RHC18 gene of S.cerevisiae whereas an identity of 47% is scored over a stretch of 53 amino acids surrounding the DA-box. A similar comparison with the rad18 gene of S. pombe reveals 47% identity over a stretch of 106 amino acids in the amino terminal end of the protein and 54% identity over a stretch of 53 amino acids in the DA-box conserved motif around the carboxyl terminal region of the protein. No homologues sequences from higher plants are found in the databases searched.

### Example 4

Complementation and Overexpression Experiments Complementation

Complementation of the mim mutant is performed by transformation of the mutant Arabidopsis line with the wild type MIM gene including its promoter and polyadenylation signal.

The mutant mim Arabidopsis line contains T-DNA comprising a nptll and bar marker gene under the control of nos and CaMV35S promoters, respectively. Therefore a new binary vector p1′hygi6, derived from p1′hygi by modification of the multiple cloning site, is used for transformation. The vector is a derivative of p1′barbi which proved to be highly efficient in Arabidopsis transformation (Mengiste et al, Plant J 12: 945-948, 1997) and has hygromycin as a selectable marker. P1′hygi can be obtained in the following way. In p1′barbi the EcoRl fragment containing the 1′promoter, bargene coding region and CaMV 35S polyadenylation signal, is inverted with respect to the T-DNA borders by digesting the plasmid with EcoRl and re-ligation. In the resulting plasmid the 1′promoter (Velten et al, EMBO J 3: 2723-2730, 1984) is directed towards the right border of the T-DNA. This plasmid is restriction digested with BamHI and NheI, and the bar gene and CaMV 35S polyadenylation signal are replaced by a synthetic polylinker sequence containing restriction sites for BamHI, HpaI, ClaI, StuI and NheI. The resulting plasmid is restriction digested with BamHI and HpaI and ligated to a BamHI-PvulI fragment of pROB1 (Bilang et al, 1991) containing the hygromycin-B-resistance gene hph linked to the CaMV 35S polyadenylation signal. The T-DNA of the resulting binary vector p1′hygi contains the hygromycin resistance marker gene under the control of the 1′promoter and the unique cloning sites ClaI, StuI and NheI located between the marker gene and the right border sequence. An oligonucleotide linker harbouring Nhe I, SpeI, XhoI, and Afl II restriction sites is inserted into the Nhe I site of the p1′hygi vector resulting in plasmid p1′hygi6 which is used to insert the wild-type MIM gene. The pBluescript phagemid pMIM 3′8.1 harbouring the 3′ end of the MIM genomic clone is restriction digested with SexAI and KpnI. The genomic fragment excised is inserted into the plasmid containing the 5′ genomic sequences of MIM (pMIM5′#1) giving pMIM5′#1.2. The remaining 3′end of the MIM gene in pMIM3′8.1 is excised as KpnI-ApaI fragment and inserted into pMIM5′1.2 creating plasmid pMIM, harbouring the MIM genomic sequence including about 2 kb of the upstream sequence. pMIM is restriction digested with Sal I, the fragment containing the MIM sequences is purified by agarose gel electrophoresis and subsequently ligated into the XhoI site of XhoI-cut and dephosphorylated p1′hygi6. The resulting construct is introduced by direct transformation into Agrobacterium tumefaciens strain C58ClRif^{R} containing a nononcogenic Ti plasmid (pGV3101) (Van Larebeke et al, Nature 252: 169-170, 1974). T-DNA containing the wild-type MIM gene is introduced into mim mutant plants by the method of in planta Agrobacterium mediated gene transfer (Bechtold et al, C R Acad Sci Paris, Life Sci 316: 1194-1199, 1993). Seeds of infiltrated plants are grown on hygromycin-containing medium and screened for transformants. The progeny of selfed hygromycin resistant plants are analyzed for segregation of hygromycin resistance. The families in which a 3:1 segregation ratio is observed are used for the isolation of homozygous lines bearing the newly introduced T-DNA inserted at a single genetic locus. The hygromycin resistant lines obtained are analyzed by northern blot analysis for the restoration of MIM expression. They are tested for restoration of wild type levels of MMS, UV, and temperature resistance and wild type levels of root growth. The progenies of seventeen independent transformants resistant to hygromycin and bearing the newly introduced T-DNA are examined for mim phenotypes. The phenotype of twelve of these lines reverts to the wild type in MMS, UV, X-rays and MMC sensitivity tests. The normal root growth and thermo-tolerance is also regained further supporting that the mim phenotype is caused by the lack of MIM gene product.

Overexpression

The MIM cDNA clones obtained by different methods were combined into a single vector (pCR2.1, Invitrogen) using standard cloning protocols to establish the entire MIM cDNA in a single DNA fragment. For overexpression of MIM cDNA in wild type Arabidopsis plants the entire MIM ORF is cloned under the control of the 35S CaMV promoter and NOS termination signal. The binary vector p1′hygi6.1 is used to insert a NheI-XbaI fragment containing the MIM cDNA in the sense orientation with respect to the 35S promoter of CaMV. Wild type plants of Arabidopsis are transformed with this construct. Phenotypes of plants overexpressing the MIM protein are studied. Northern blot analysis made on 16 independent lines generated with a 35S::MIMcDNA construct are analyzed. The transcript level in three selected lines is increased as compared to the wild type level of MIM expression observed in seedlings. Said lines are further analyzed for homologous recombination activity.

### Example 5

Analysis of Recombination in the Mutant

A non-selective assay system enabling visualization of intrachromosomal homologous recombination events is used. The assay system employs a disrupted chimeric β-glucuronidase (uidA) (GUS) gene (Jefferson et al, EMBO Journal 6: 3901-3907, 1987) as a genomic recombination substrate having an overlapping GUS sequence of 1213 bp in direct orientation. Said substrate is stably integrated in an Arabidopsis line used for the recombination assay and is further on referred to as N1DC1. Upon intrachromosomal homologous recombination expression of the GUS gene is restored. Cells in which recombination events occur can be evaluated upon histochemical staining of the whole plant seedling.

The mim mutant line is crossed to a line of Arabidopsis C24 ecotype (N1DC1 no.11) which is transgenic for the recombination substrate (Swoboda et al., EMBO Journal 13: 481-489, 1994). Line N1DC1 no.11 contains two copies of the recombination substrate at a single locus. F1 plants of the crosses are allowed to self-pollinate. Progeny of said F1 plants are plated on nutrient medium and plants with short roots, that is plants which are homozygous for the mim mutation, are selected and grown to maturity. Progeny of these F2 plants are selected on 10 mg I⁻¹ phosphinotricin (ppt) and 10 mg I⁻¹ hygromycin. Lines homozygous resistant to ppt, that is plants homozygous for the mim mutation, and resistant to hygromycin, that is plants homozygous for the recombination substrate, are used for the intrachromosomal recombination assay. For comparison recombination events are also assayed for plants of (a) wild type (Wassilewskija ecotype), (b) line N1DC1 no.11 (C₂₄ ecotype), and (c) Segregating F3 plants from the same crosses mentioned above having the genotype of Line N1DC1 no. 11 and the wild type parental ecotype of the mutant (Wassilewskija) to exclude the contributions of ecotype on recombination. The histochemical (X-gluc) assay is performed as described by Jefferson et al supra. Recombination frequency in the mutant (mim) background is found to be 3.9 fold lower than in the wild-type genetic background.

What is claimed is:

1. DNA comprising an open reading frame encoding a protein characterized by an amino acid sequence having 30% or more identity with SEQ ID NO: 3

2. The DNA according to claim 1 comprising an open reading frame encoding a protein comprising a stretch of 100 or more amino acids with 50% or more sequence identity to a stretch of aligned amino acids of a protein member of the SMC protein family.

3. The DNA according to claim 1, wherein the open reading frame encodes a protein characterized by the amino acid sequence of SEQ ID NO: 3

4. The DNA according to claim 1 characterized by the nucleotide sequence of SEQ ID NO: 1 or SEQ ID NO: 2.

5. The DNA according to claim 1, wherein the open reading frame encodes a protein contributing to recombination repair of DNA damage in a plant cell.

6. The DNA according to claim 1, wherein the open reading frame encodes a protein conferring hypersensitivity to treatment with methyl methanesulfonate (MMS).

7. The DNA according to claim 6, wherein the open reading frame encodes a protein conferring hypersensitivity to treatment with X-rays, UV light or mitomycin C.

8. The DNA according to claim 1, wherein the open reading frame encodes a protein with a NTP binding region followed by a first coiled coil region, a hinge or spacer, and a second coiled coil region followed by a C-terminal DA-box which harbours a Walker B type NTP binding domain.

9. The protein encoded by the open reading frame of any one of claims 1 to 8.

10. A method of producing DNA according to claim 1, comprising
screening a DNA library for clones which are capable of hybridizing to a fragment of the DNA defined by SEQ ID NO: 1, wherein said fragment has a length of at least 15 nucleotides;
sequencing hybridizing clones;
purifying vector DNA of clones comprising an open reading frame encoding a protein with more than 40% sequence identity to SEQ ID NO: 3
optionally further processing the purified DNA.

11. A polymerase chain reaction, wherein at least one oligonucleotide used comprises a sequence of nucleotides which represents 15 or more basepairs of SEQ ID NO: 1.


SEQUENCE LISTING (15 sequences)

SEQ ID NO: 1 - DNA, 10156 bp, Arabidopsis thaliana
  misc_feature (1)..(10156): Wild-type MIM gene “n”= A + T + G + C
gattttcatcagaatctatttcgatatagttttcagtatcttttctttttcgagttgataccaaactatcaatcgatttcagattctgaagatattctgacatgttgtcatccttcatttgtaaaagacataaagcactttcttcaatagttatatcgctttcagactctatcgaattttcctcttccaaatccacgacgatctttttcttgacagttgttcgttgctcagcttctttgtttagatctggctttggaccaccaacaacttcactggtgtggacaaatctcgccagaactgtttcattaggtcttctaaagcttgctccaaggttgttgcatgatcgtagtgatggagtgatcgagccagttgatgaagatgatccagggaaaaatttcaaagtgtgagctctcatgtgaccaccaagagttttccccgtttngaaattgttttctttgcacaccttgncaggtttttgttttcctaggangtgantacattttggacggttgaaaaacccaaaaaaaaaactaccaaatttttaggcgttaaagattttgattgctttttaatgcggaaaagtgtttgtgaatattatgtgattttgaatccagtggagatactcatatatatatataggatgcatgagagggaggaacacaatttctgttcaaaaggagttaaccacttaacataagtgtttgttcattatgttctcacatttagttacaagcatattttattctggtcaaaaaaaacaaagtcaacaattatatacaagctaatcttttattttcttactctcttttttttaaaatagtcgtcgtttaggatttttacataaaagttaagaaaacaattaaattttttatttatttttattggttacgacattgaacaataaggatattattgaaagttttatcaaatatttatattgaaaatctaaaatgacgattattacgaattaaaatttttagtgttaggaaggacaatccaaattaaaacggaaaaagtatataaaaaaagtaacagtagttttttcgtgttttatacaaataactatagataatttaacgtataaattataatcgaatgtatttgaatcgaacaacgtgaacatgataggaatgtgcatgatatttccggaaaattatgcacaatatctgaaaatctatttaatcacaccgtaaaacaaatacacttttgtagtataaaattattttaatttagttaagattttaattttttttctttcttacagtgaacactactgttgaccaaaagaaaaagggtctattgggctaaaaacaactgtagctaatgggccatattagggggactttaaggcccattggtgttcggtcaataagatcttggagatcatcatcatcgtacggtaaaagacaagcggaatatacaacggggaacgaacaatagcaatctctttcccgccctaagcagtcgcatcaatggagcttgctctattctaatttgtttcaaccgagtgagagaagaaaccctagaacgcgaaaagccatggtaaaatctggagctcgagccagtgattcattcatcaaacaacgttctgggtcgggttcgattctgaggatcaaagttgagaatttcatgtgccatagttatctccagattgagtttggcgagtgggttaatttcatcaccggccaaaacggaagtaagtcttctccttctgtttaaaaaaatgttttttagagctctgattgactgaatttaatcacgcatgccttattgggaattgggtttcgcctaattttgatatcccagatttttcaatttgattcaattgtgttcaactattcaggttaggaggtagtggaaagtcttttttatttatttagaattggtttntncacagtcaatgaccaagagttttaatntggacttttgattaaaaatcttaggtggtaagagtgcaatactgactgcactatgtattgcatttggatgtcgagcgagagggactcagcgtgctgccactctaaaggatttcattaaaactggatgcaggttttgtacacttgcactgtgttttgtctaaatatcagatttgcagattggaagtgaaaataggacatgtttagtggcgcttattcttctttcttaaattttttagattgtctcttgtcattgattaggagatgactaatgataagagtgactgaaattcctttccaatttggttggatttctttgcagctatgccgttgtccaagtagaaatgaaaaacagtggagaggatgcttttaagtctgaaatttatggtggcgttataattatcgaacgcaggataactgagtctgctacagctactgttctcaaagattatctaggttaattcattgtactctctataataatttatagtttgacttacagtttatctcatagcccccgtgtttgttgtgatgcctgtctccgttcttattttcttctccaacaactctatctttgtgttatgtgcatatatataaaactattgatttaaaatgtttttctgatttcttattttctgcaggaaaaaaagtaagtaacaaaagggacgagctacgggaacttgttgaacattttaatgtgagttttggccgttcattcaaaattttagaaagtattaagtgatatcagatacttggcatactcagtactgtattcttattatattttacatgtgtagattgatgttgaaaatccgtgtgtggtaatgagtcaagacaaagcagggagttcttacattctggaatgcaaaggtaaattcaaggtatgtcacgtgaattgatatatatatcaaagtcaaccatgttgttattatggctgaaaattttcgctctcaagttcttttttaaggaaccttcttcagcaagtcaatgatcttctccaaagtatctacgaacacttgacaaaagcaactgctatagtcgatgaattggagaacacaattaaaccaatagaaaaggagatcagtgagttgcgtggaaagataaagaatatggaacaagttgaagaaatagctcaaaggttgcagcagttgaagaagaaactggcttggtcatgggtatatgatgtgggtaggcagctccaggaacagactgagaagattgtgaagcttaaagaacgtataccgacttgccaagctaaaatagattgggaactggtaagtaatacatactttccttcatccgaaatttggatggctacaaaaatcgaaaggtaaagatgctgggtgcattacaagttgtaacttctctctgatatcctacctggccatcataagataaaaatggagtttttagctgtataaaagagagagtttgattatgtagagtcttgtggtattcctaacgtaaactcctcattgggcatagttaatgtgtgccatattggttccatatgtttatgtgaggttttgcctctaacatgttcaattttcttagagcgcaaaccttcgcctgccttactgtaggggtctactgatgagtgaatgattgcttaattcatgtttcgagctcagactggttgattatcattgacctttttgtagggaaaagtggaatcattaagggatacgttgaccaagaagaaagctcaagttgcgtgtctgatggatgaatcaactgcaatgaagagagagatagagagttttcaccaatcagccaagacggtttgtaaacttagttttaagagggatctagtgggtggaattttgcctaaagaattgacaaattatcttcccttatttaacaaaatatattttcttgttcagtgtgaancaaataaaaatnctggattttggcaaatgggctgcaaggctctaatatgcttattattagtttanattaattttgaaaagttgctttcgggtataanttaattgtccacatcttgttatgttgtgttccttgaaaaaatctttgtgtgttcctattttaaggctgtacgagaaaaaattgccctacaagaagaattcaatcataagtgcaattatgttcaaaagattaaggatcgtgttagaaggcttgaacggcaagttggagatatcaatgaacagacaatgaagaacacacaggtccccaatatcagtcacatatcttaaaaaggaaaaactatgtcatgtttcttttgtgctgagtgtcttggcttaacgatcaagatattgtgaaggtgtgtatacatggacagatatacttgtgcatatttctcataggttgataattatgtaggctgaacaatctgaaatcgaggagaaactaaaatatttggagcgggaggttgagaaagttgaaacattgcgttccaggttatgatcttaagttttctgtttccttttcgtctgcttagcatttcggagtcttctctctcgtctttaaacatgttttaaagattgatactttagattgaaagaggaagagaactgcttcttggaaaaagcgtttgaagggaggaaaaagatggaacacatcgaggatatggtacaactatacctattacttacacatgaatctgaagtcttttttttcaatcagtttcgcaggttgtttacaattgcaattaacacctcaatcttttcgttgtcctttttttgcttctagattaaaaaccatcaaaagaggcaaagattcataacctcgaacattaatgatctgaagaaacatcaaacaaataaggtgcatcatagttatttcatcacaaaatatagtgtttcaactggatcttgtcaagcctctttcggaaatatctaagaggcatacaaatacatacatgctcaccaggttactgcatttggaggggacagagtcattaatcttctgcaggctattgagagaaatcatcgtagatttagaaaaccaccaattggtcctattggctcccatgtggtaagtttctactcgtttcgtttgcaatctgtgcaccaaacaaactatttcacttgttatcttatattgacatgtgcaactgttgctgtaacaatcttttgatgtgaacattttgtgggttaaaaagtctcttctaatggtgtgatgttctgcagactttagtcaatggcaataaatgggcttcttcagttgaacaagctcttggaaccctattaaatgccttcattgtgactgatcacaaagattctctcactctaagaggctgtgcgaatgaagctaactatagaaatcttaagattatcatctatgacttttcgagaccaaggttcaaactcgaaataagcattttcatatttccttttaaccatctgcattgatgaatgggttnctttataacgcaaatatttgctatctcttcatttatgcaggttaaatataccaaggcacatggtgcctcagacagaacacccaactatattctctgtcatagactctgataacccaaccttccttaatgtcttggtggatcaggtttgtactttcaaattttccctccacttactaaatttcttcattcttacagccttttatgacggtgttcatattttagtcttgttttctgaatatttcagagtggtgttgagaggcaagtgcttgcagaaaattatgaggagggaaaggcggttgcatttgggaaaaggctctcaaatctgaaggaggtttacactttagacggatacaaaatgtaagtaatttgttagatttggcatggacaatcccgctatatctctccccttgcaaacaaaaacaaatcctttgcatggcgggggataatcttttcttttgaaatagaattttgaacaatgacatgcacccctctttctgtatcctggctctggatttctccaatatcaattttccaccataagacaaacaaaaagcttcaaacaatagaactttttgtttgatatatttcattttcaaaatcccttcaacttatctttgaagggacccagaatatganatatcccgactgtctttgcatctacaacatctacaatatcttagtccttgttaaaataaaattcatttttaatttaaaanggatttgacttgaaaacntctaagggatataagaatatccncccccatccataaccctaatttttctaattnttaccccaggtttttttcgtgggccagttcagactactcttcctcctctttctcgtagaccttcgcgactctgtgcttcttttgatgaccagatcaaggatcttgaaatagaggcttcaaaagaacaaaacgagataaatcaatgcatgagacgtaagagggaggcagaggagaatcttgaggaacttgagttgaaagtgcgccaactggaggtattgtctcattgattaatccagtagaaactagagttcccagtctttatatatcttaactgaaatgattagcttgaatttacaaagctttagttcgctagtatccataaccgtttccatatttttctttgttcggcttgtgaagaagcaccgcagccaagcagagaaggttttgacgacaaaggaacttgagatgcacgatttgaagaatacagtcgctgctgagatcgaatcattaccttcttcaagtgttaatgagcttcaacgtgaaatcatggttagtttgttggaatttttctttattgttactgttttcgccttctcctcatgtattttttcttcttttctaattaatatgccagaaagacctagaagagatagatgagaaagaagctttccttgagaagctccaaaactgcttgaaagaagctgagctaaaggctaataaacttacagctttatttgagaacatgcgtggtatgtgtgtgatagaccactgtttctgaaacacctgtatctttttcttgtggtgtggtcgaacatgaatagatcttcttgattgagtggccgactctattcttactttttctcaaaacgtgcatgtagagtcagccaagggtgaaattgatgcctttgaggaagcagagaatgagctaaagaagattgagaaagaccttcagtctgccgaagcggtaccccttcttttttcgacgtgaagattttttttacttggtttgcatgtagaatgtaagcttgattctgtttcaggagaaaatccattacgagaacataatgaaaaacaaggtcctacctgatattaagaatgctgaggctaactacgaggagcttaaaaataagcgaaaggtacatataataagcaattactcagaaatttttcgaaatgacatttgcaacttctttccgttgtatacaacacacacacacacaatatatatatatatatatatatagatacctctcttaatctcttgtaaaccctctctaaggaaatggccagtttgaaacccggtatgacatgttgtctagtctatggaacttagctactggagtaatgtgtattagctgtagaatttttttatcttgtaggttattattgcccagggttcatttttctgtggttatgaattgcatgtgctgaaatttcaggaaagtgaccagaaggcctctgaaatttgtcctgagagtgagatagaatctttgggtccctgggatgggagtactcctgagcaactcagtgctcagattaccagaatgaatcagagacttcatcgagagaatcagcagtgcgtattttaatattgcctttcagcttttttccttcacacagaaacgacctgtgacagtaaattacgtgcttcaattttgttcgtgcaggttttctgaatcaattgatgaccttaggatgatgtatgagagcctagaacgaaagattgcaaagaagcgcaaatcctatcaagaccatcgagaaaaactcatggttgagtctgcagatctgtttatgttaaagctctttgtgttgttatcgtattatcgtttaatgtattcatcactatttgatcaggcctgcaaaaatgctctagattcacggtgggccaaatttcaaagaaatgcatctcttcttcggcgccagttaacatggcagtaagagtcgctttccccattgccacctacatagataaatctgtagttcggttgtctcttgagattagtatgatttttttttccatatggagctttctttgacgttaatcttctaagcagattcaacgctcacttgggaaagaaaggtatcagcggacacatcaaagtcagttatgaaaataaaactttgtccatagaagttaattgacactgccatggtacggtttttgctactagcgtgcccatattattacgtcatctgattgatatcgttctttatgacaggttaaaatgcctcaagacgcaacaagcaatgtcgttcgagacaccaaaggtctttcaggtactgcatccttccacactcttaaaaatcatacatctgattcattgccatataaagacatttcctatgtgtaacgctcttctcattatactaggcggagaacgttctttctcaactttatgttttgcactagctcttcacgagatgacagaagccccgtttcgagcaatggatgagtttgatgtgtttatggtattatgtccttttaagaattctctcttttacgagctttacgttgggatgagactaacatttttaactttctgattctgaaatataggatgcagtcagtcggaaaattagcttggacgcactggtggattttgcaattggagaaggatcgcagtggatgttcatcacccctcatgatatcaggtaaccaaccgatcaatttcaaaaaccatggaactcagtctgtgaagtgaataacttggatgaaactctttatctcttgtgctcttttacagcatggtgaagtcgcacgagaggataaagaaacagcaaatggctgctcctcgttcttgaaaacaaaaaaaaactctccttgtatagctccataaaggaacacacaattttgcttggcatgacccattcaagcattttatgttttgtgtctgcatttttcgccagttctcacttatgtgtttctttacgggactcatcagggcatcctcggctttgagtcaatactacgacgagctgatgggaagtttgagaagagctttgtcatccatggtcatggtgtattgatttgaatttacaggtgctgagtgagacccttttttgttttctctcatttctttttatgacatttatatattgtcaaactttgattttaaaactgtattatatatcatttttattgacttataattatcatttttagatcgaccaagtgtgacagattactttcctagtgataaagactagtgattgaggacaatagcagaaagactaagtaacttttaagcttcggattaagaaatgtccatagtttctgattctttttggcaagtttaagtacatcctgaatgttcgataaaggctagtgtcatcaacatctctaatacatatgacagccaaggagcatccattgaaaaatgaggcaataaaattgagtacttataaactgttcgggggntgggttattcgnaattanttcgntttaaaggntatttncatccaactaatttggatccagggccctgccaaaaaatggtttttaggattncacggnggggcccaaaattttcaaaaggaaatgcatttnttttttcgggcgccagttaaacatggcagtaagagtcgctttcccccattgccacctaacatagataaatctgtagttcggttgtcttcttgagattagtatgatttttttttcccatatggagctttctttgacgttaatcttctaagcagattcaacgctcacttgggaaagaaaggtatcagcggacacatcaaagtcagttatgaaaataaaactttgtccatagaggtaattgacactgccatggtacggtttttgctactagcgtgcccatattattacgtcatctgattgatatcgttctttatgacaggttaaaatgcctcaagacgcaacaagcaatgtcgttcgagacaccaaaggtctttcaggtactgcatccttccacactcttaaaaatcatacatctgattcattgccatataaagacatttcctatgtgtaacgctcttctcattatactaggcggagaacgttctttctcaactttatgttttgcactagctcttcacgagatgacagaagccccgtttcgagcaatggatgagtttgatgtgtttatggtattatgtccttttaa

SEQ ID NO: 2 - DNA, 3668 bp, Arabidopsis thaliana
  misc_feature (2)..(3668): MIM cDNA
catcaatggagcttgctctattctaatttgtttcaaccgagtgagagaagaaaccctagaacgcgaaaagccatggtaaaatctggagctcgagccagtgattcattcatcaaacaacgttctgggtcgggttcgattctgaggatcaaagttgagaatttcatgtgccatagttatctccagattgagtttggcgagtgggttaatttcatcaccggccaaaacggaagtggtaagagtgcaatactgactgcactatgtattgcatttggatgtcgagcgagagggactcagcgtgctgccactctaaaggatttcattaaaactggatgcagctatgccgttgtccaagtagaaatgaaaaacagtggagaggatgcttttaagtctgaaatttatggtggcgttataattatcgaacgcaggataactgagtctgctacagctactgttctcaaagattatctaggaaaaaaagtaagtaacaaaagggacgagctacgggaacttgttgaacattttaatattgatgttgaaaatccgtgtgtggtaatgagtcaagacaaagcagggagttcttacattctggaatgcaaaggtaactcaagttcttttttaaggaaccttcttcagcaagtcaatgatcttctccaaagtatctacgaacacttgacaaaagcaactgctatagtcgatgaattggagaacacaattaaaccaatagaaaaggagatcagtgagttgcgtggaaagataaagaatatggaacaagttgaagaaatagctcaaaggttgcagcagttgaagaagaaactggcttggtcatgggtatatgatgtgggtaggcagctccaggaacagactgagaagattgtgaagcttaaagaacgtataccgacttgccaagctaaaatagattgggaactgggaaaagtggaatcattaagggatacgttgaccaagaagaaagctcaagttgcgtgtctgatggatgaatcaactgcaatgaagagagagatagagagttttcaccaatcagccaagacggctgtacgagaaaaaattgccctacaagaagaattcaatcataagtgcaattatgttcaaaagattaaggatcgtgttagaaggcttgaacggcaagttggagatatcaatgaacagacaatgaagaacacacaggctgaacaatctgaaatcgaggagaaactaaaatatttggagcgggaggttgagaaagttgaaacattgcgttccagattgaaagaggaagagaactgcttcttggaaaaagcgtttgaagggaggaaaaagatggaacacatcgaggatatgattaaaaaccatcaaaagaggcaaagattcataacctcgaacattaatgatctgaagaaacatcaaacaaataaggttactgcatttggaggggacagagtcattaatcttctgcaggctattgagagaaatcatcgtagatttagaaaaccaccaattggtcctattggctcccatgtgactttagtcaatggcaataaatgggcttcttcagttgaacaagctcttggaaccctattaaatgccttcattgtgactgatcacaaagattctctcactctaagaggctgtgcgaatgaagctaactatagaaatcttaagattatcatctatgacttttcgagaccaaggttaaatataccaaggcacatggtgcctcagacagaacacccaactatattctctgtcatagactctgataacccaaccttccttaatgtcttggtggatcagagtggtgttgagaggcaagtgcttgcagaaaattatgaggagggaaaggcggttgcatttgggaaaaggctctcaaatctgaaggaggtttacactttagacggatacaaaatgttttttcgtgggccagttcagactactcttcctcctctttctcgtagaccttcgcgactctgtgcttcttttgatgaccagatcaaggatcttgaaatagaggcttcaaaagaacaaaacgagataaatcaatgcatgagacgtaagagggaggcagaggagaatcttgaggaacttgagttgaaagtgcgccaactgaagaagcaccgcagccaagcagagaaggttttgacgacaaaggaacttgagatgcacgatttgaagaatacagtcgctgctgagatcgaatcattaccttcttcaagtgttaatgagcttcaacgtgaaatcatgaaagacctagaagagatagatgagaaagaagctttccttgagaagctccaaaactgcttgaaagaagctgagctaaaggctaataaacttacagctttatttgagaacatgcgtgagtcagccaagggtgaaattgatgcctttgaggaagcagagaatgagctaaagaagattgagaaagaccttcagtctgccgaagcggagaaaatccattacgagaacataatgaaaaacaaggtcctacctgatattaagaatgctgaggctaactacgaggagcttaaaaataagcgaaaggaaagtgaccagaaggcctctgaaatttgtcctgagagtgagatagaatctttgggtccctgggatgggagtactcctgagcaactcagtgctcagattaccagaatgaatcagagacttcatcgagagaatcagcagttttctgaatcaattgatgaccttaggatgatgtatgagagcctagaacgaaagattgcaaagaagcgcaaatcctatcaagaccatcgagaaaaactcatggcctgcaaaaatgctctagattcacggtgggccaaatttcaaagaaatgcatctcttcttcggcgccagttaacatggcaattcaacgctcacttgggaaagaaaggtatcagcggacacatcaaagtcagttatgaaaataaaactttgtccatagaggttaaaatgcctcaagacgcaacaagcaatgtcgttcgagacaccaaaggtctttcaggcggagaacgttctttctcaactttatgttttgcactagctcttcacgagatgacagaagccccgtttcgagcaatggatgagtttgatgtgtttatggatgcagtcagtcggaaaattagcttggacgcactggtggattttgcaattggagaaggatcgcagtggatgttcatcacccctcatgatatcagcatggtgaagtcgcacgagaggataaagaaacagcaaatggctgctcctcgttcttgaaaacaaaaaaaaactctccttgtatagctccataaagggcatcctcggctttgagtcaatactacgacgagctgatgggaagtttgagaagagctttgtcatccatggtcatggtgtattgatttgaatttacaggtgctgagtgagacccttttttgttttctctcatttctttttatgacatttatatattgtcaaactttgattttaaaactgtattatatatcatttttattgacttataattatcatttttagatcgcccaagtgtgacagattactttcctagtgataaagactagtgattgaggacaatagcagaaagactaagtaacttttaagcttcggattaagaaatgtccatagtttctgaaaaaaaaaaaaaaaaaaaaaaaaaaaaaaaaaaaaaaaaaaaaaaaaaaaaaaaa

SEQ ID NO: 3 - protein, 1055 aa, Arabidopsis thaliana
MVKSGARASDSFIKQRSGSGSILRIKVENFMCHSYLQIEFGEWVNFITGQNGSGKSAILTALCIAFGCRARGTQRAATLKDFIKTGCSYAVVQVEMKNSGEDAFKSEIYGGVIIIERRITESATATVLKDYLGKKVSNKRDELRELVEHFNIDVENPCVVMSQDKAGSSYILECKGNSSSFLRNLLQQVNDLLQSIYEHLTKATAIVDELENTIKPIEKEISELRGKIKNMEQVEEIAQRLQQLKKKLAWSWVYDVGRQLQEQTEKIVKLKERIPTCQAKIDWELGKVESLRDTLTKKKAQVACLMDESTAMKREIESFHQSAKTAVREKIALQEEFNHKCNYVQKIKDRVRRLERQVGDINEQTMKNTQAEQSEIEEKLKYLEREVEKVETLRSRLKEEENCFLEKAFEGRKKMEHIEDMIKNHQKRQRFITSNINDLKKHQTNKVTAFGGDRVINLLQAIERNHRRFRKPPIGPIGSHVTLVNGNKWASSVEQALGTLLNAFIVTDHKDSLTLRGCANEANYRNLKIIIYDFSRPRLNIPRHMVPQTEHPTIFSVIDSDNPTFLNVLVDQSGVERQVLAENYEEGKAVAFGKRLSNLKEVYTLDGYKMFFRGPVQTTLPPLSRRPSRLCASFDDQIKDLEIEASKEQNEINQCMRRKREAEENLEELELKVRQLKKHRSQAEKVLTTKELEMHDLKNTVAAEIESLPSSSVNELQREIMKDLEEIDEKEAFLEKLQNCLKEAELKANKLTALFENMRESAKGEIDAFEEAENELKKIEKDLQSAEAEKIHYENIMKNKVLPDIKNAEANYEELKNKRKESDQKASEICPESEIESLGPWDGSTPEQLSAQITRMNQRLHRENQQFSESIDDLRMMYESLERKIAKKRKSYQDHREKLMACKNALDSRWAKFQRNASLLRRQLTWQFNAHLGKKGISGHIKVSYENKTLSIEVKMPQDATSNVVRDTKGLSGGERSFSTLCFALALHEMTEAPFRAMDEFDVFMDAVSRKISLDALVDFAIGEGSQWMFITPHDISMVKSHERIKKQQMAAPRS

SEQ ID NO: 4 - DNA, 21 bp, Artificial sequence
  misc_feature: T-DNA oligonucleotide
ggtttctacaggacgtaacat

SEQ ID NO: 5 - DNA, 32 bp, Artificial sequence
  misc_feature: Description of Artificial Sequence T-DNA adjacent 32 nucleotides
ctgcagatctgtttatgttaaagctctttgtg

SEQ ID NO: 6 - DNA, 22 bp, Artificial sequence
  misc_feature: Description of Artificial Sequence FP1
ctgggtcgggttcgattctgag

SEQ ID NO: 7 - DNA, 23 bp, Artificial sequence
  misc_feature: Description of Artificial Sequence FP2
ggtaagagtgcaatactgactgc

SEQ ID NO: 8 - DNA, 24 bp, Artificial sequence
  misc_feature: Description of Artificial Sequence FP3
gcagctatgccgttgtccaagtag

SEQ ID NO: 9 - DNA, 23 bp, Artificial sequence
  misc_feature: Description of Artificial Sequence SP1
aatgactctgtcccctccaaatg

SEQ ID NO: 10 - DNA, 23 bp, Artificial sequence
  misc_feature: Description of Artificial Sequence SP2
atgttcgaggttatgaatctttg

SEQ ID NO: 11 - DNA, 21 bp, Artificial sequence
  misc_feature: Description of Artificial Sequence RP1
gactcagttatcctgcgttcg

SEQ ID NO: 12 - DNA, 39 bp, Artificial sequence
  misc_feature: Description of Artificial Sequence Oligo dT-anchor primer
gaccacgcgtatcgatgtcgacttttttttttttttttv

SEQ ID NO: 13 - DNA, 24 bp, Artificial sequence
  misc_feature: Description of Artificial Sequence RP2
ggacaacggcatagctgcatccag

SEQ ID NO: 14 - DNA, 22 bp, Artificial sequence
  misc_feature: Description of Artificial Sequence PCR anchor primer
gaccacgcgtatcgatgtcgac

SEQ ID NO: 15 - DNA, 24 bp, Artificial sequence
  misc_feature: Description of Artificial Sequence RP3
ggcagcacgctgagtccctctcgc